(19) United States
(12) Patent Application Publication
(10) Pub. No.: US 2021/0279984 A1
(43) Pub. Date: Sep. 9, 2021

(54) BELT DEVICE WITH SMART FUNCTION FOR CARRIER

(71) Applicant: Oxti Corporation, Taipei (TW)

(72) Inventor: Chih Feng HO, Taipei (TW)

(21) Appl. No.: 16/808,485

(22) Filed: Mar. 4, 2020

(51) Int. Cl. G07C 9/00; G16H 20/30

(52) U.S. Cl. CPC G07C 9/00857; A61B 5/486; G16H 20/30

(57) ABSTRACT

A belt device combination includes a carrier member for supporting a user, a strap member for engaging with the user, the strap member includes one end portion attached to the carrier member, and a lock member is attached to the carrier member, a buckle member is attached to the other end portion of the strap member for engaging with the lock member and for anchoring the user to the carrier member, and a controlling device includes a processing module connected to the lock member for releasing the buckle member from the lock member selectively, and a timing module connected to the processing module for controlling a releasing time interval of the buckle member from the lock member.

### BACKGROUND OF THE INVENTION

### 1. Field of the Invention

The present invention relates to a belt device combination, and more particularly to a smart or intelligent belt device combination including an improved and smart or intelligent structure or configuration for posture correction purposes and for reminding the user that he has kept a sedentary posture for a long time and that he is required to do some physical exercise.

### 2. Description of the Prior Art

Various kinds of typical smart belt devices have been developed and provided for public safety purposes and for maintaining power to these devices on a long term basis, throughout the shift of the public safety employee, and comprise a structure or configuration for generating data, which must be captured and stored, and/or which must be transferred or uploaded either immediately or at a later time.

For example, U.S. Pat. No. 9,859,938 B2 to Piccioni, U.S. Pat. No. 10,046,732 B2 to Piccioni, U.S. Pat. No. 10,112,575 B2 to Piccioni, U.S. Pat. No. 10,398,355 to Kang et al., and U.S. Pat. No. 10,514,237 B2 to Piccioni disclose several of the typical smart belt devices provided for public safety purposes and for generating data and for maintaining power to these devices on a long term basis, throughout the shift of the public safety employee or users.

However, the typical smart belt devices may not be provided for reminding the user that he has kept a sedentary posture for a long time and that he is required to do some physical exercise for healthy purposes.

The present invention has arisen to mitigate and/or obviate the afore-described disadvantages of the conventional belt devices.

### SUMMARY OF THE INVENTION

The primary objective of the present invention is to provide a belt device combination including an improved and smart or intelligent structure or configuration for posture correction purposes and for reminding the user that he has kept a sedentary posture for a long time and that he is required to do some physical exercise.

In accordance with one aspect of the invention, there is provided a belt device combination comprising a carrier member for supporting a user, a strap member for engaging with the user, the strap member including a first end portion attached to the carrier member, and a second end portion, a lock member attached to the carrier member, a buckle member attached to the second end portion of the strap member for engaging with the lock member and for anchoring the user to the carrier member, and a controlling device attached to the lock member, the controlling device including a processing module connected to the lock member for releasing the buckle member from the lock member selectively, and a timing module connected to the processing module for controlling a releasing time interval of the buckle member from the lock member.

The controlling device includes an input module connected to the timing module. The controlling device includes a remote module connected to the processing module.

The carrier member is selected from a chair, the first end portion of the strap member is attached to a first side portion of the carrier member, and the lock member is attached to a second side portion of the carrier member.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is a perspective view illustrating the operation of a belt device combination in accordance with the present invention;

FIGS. 2, 3 are other perspective views similar to FIG. 1, illustrating the operation of the belt device combination;

FIG. 4 is a further perspective view similar to FIGS. 1-3, illustrating the operation of the belt device combination;

FIGS. 5, 6 are still further perspective views similar to FIGS. 1-4, illustrating the other operation of the belt device combination;

FIGS. 7, 8 are still further perspective views similar to FIGS. 1-6, illustrating the further operation of the belt device combination;

FIG. 9 is a still further perspective view similar to FIGS. 1-8, illustrating the still further operation of the belt device combination; and

FIG. 10 is a block diagram illustrating the parts or elements for actuating or operating the belt device combination.

### DETAILED DESCRIPTION OF THE PREFERRED EMBODIMENT

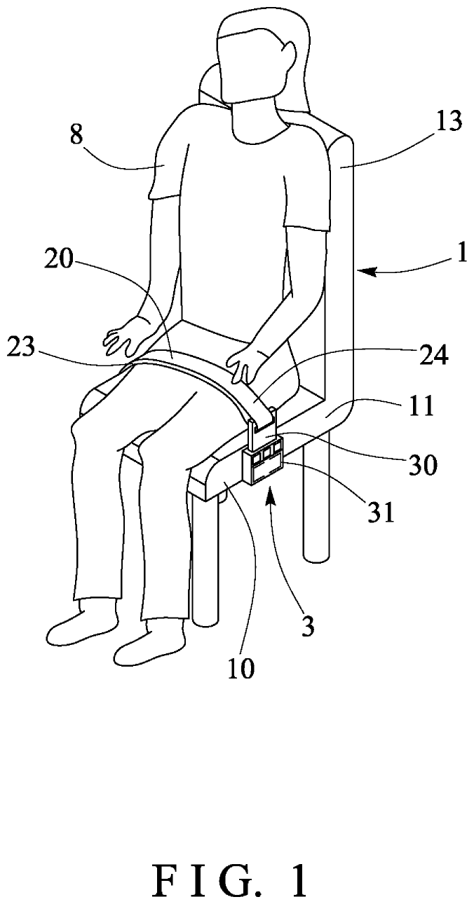

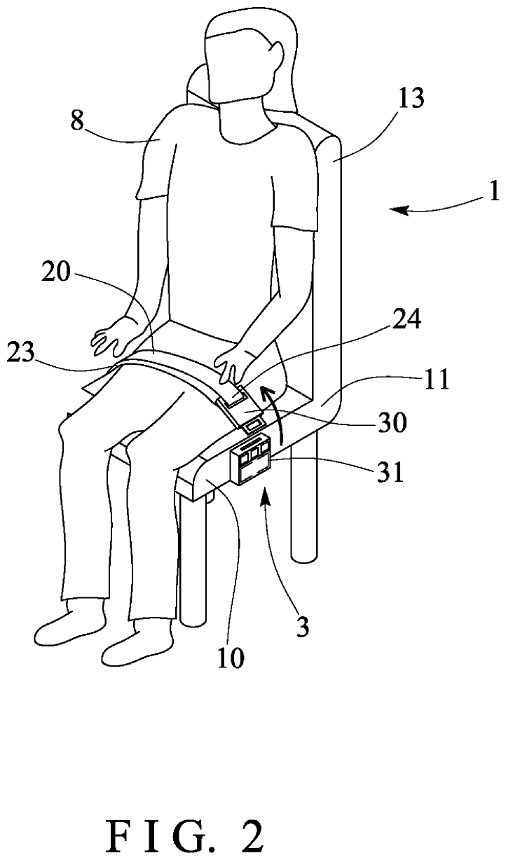

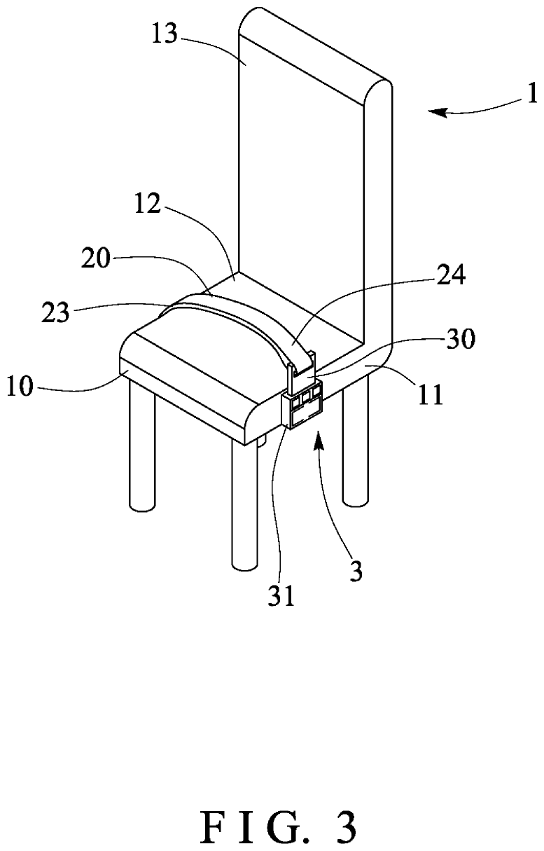

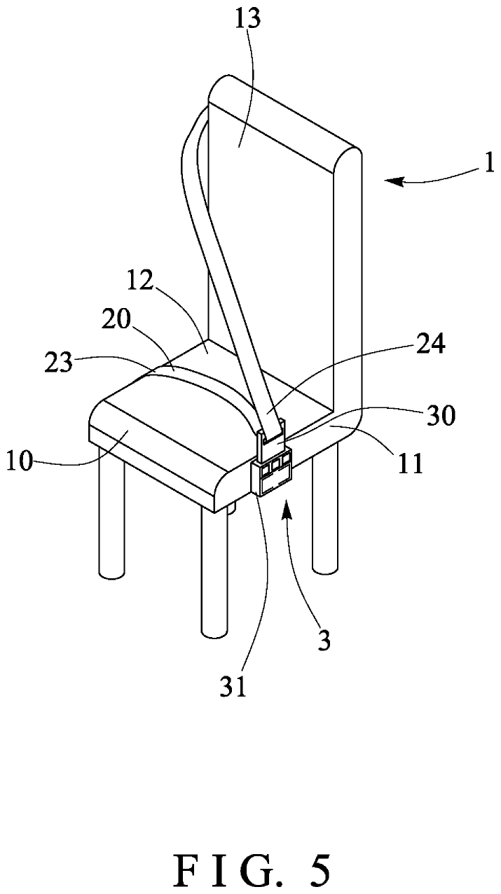

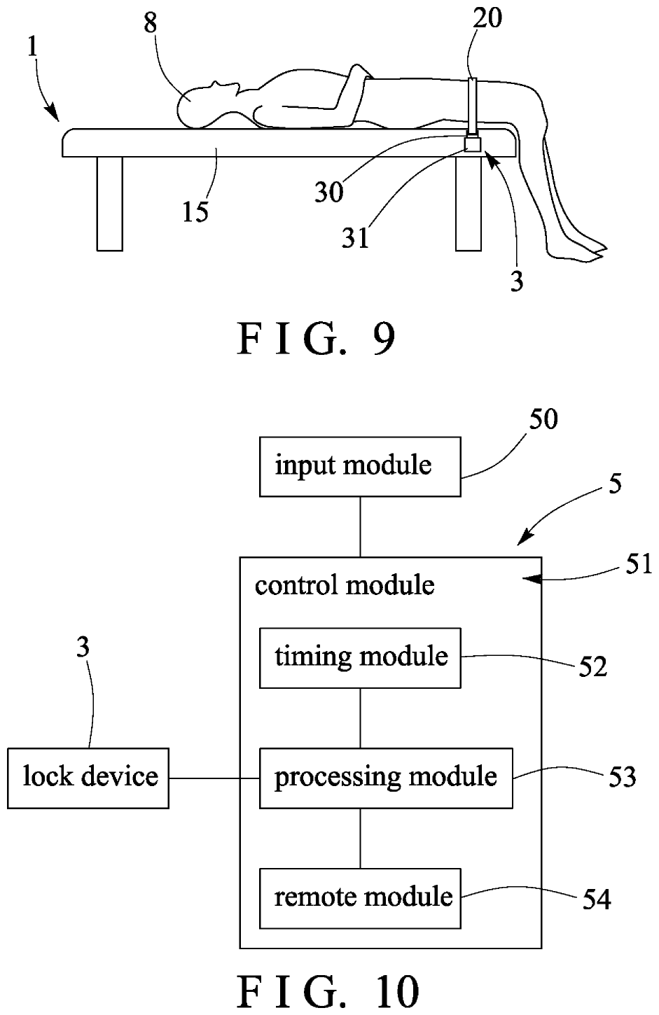

Referring to the drawings, and initially to FIGS. 1-3, a belt device combination in accordance with the present invention comprises a carrier device 1 which may be selected from a carrier member, such as a chair 10 (FIGS. 1-8), a bed 15 (FIG. 9) or the like for supporting a user 8 thereon, for example, the carrier member or chair 10 of the carrier device 1 includes a first side portion 11 and/or a second side portion 12, and/or a seat back 13 for engaging with and for supporting the user 8 on the carrier device 1.

One or more belt or strap members 20, 21, 22 are provided for engaging with the user 8 (FIGS. 1-2, 4, 6, and 8) and for anchoring or retaining or positioning or supporting the user 8 on the carrier device 1. The strap members 20, 21, 22 each include one or first end portion 23 attached or mounted or secured to such as the first or the second side portion 12 of the chair 10 or of the carrier device 1, and a buckle member 30 of a lock device 3 is attached or mounted or secured to the other or second end portion 24 of the strap member 20, 21, 22.

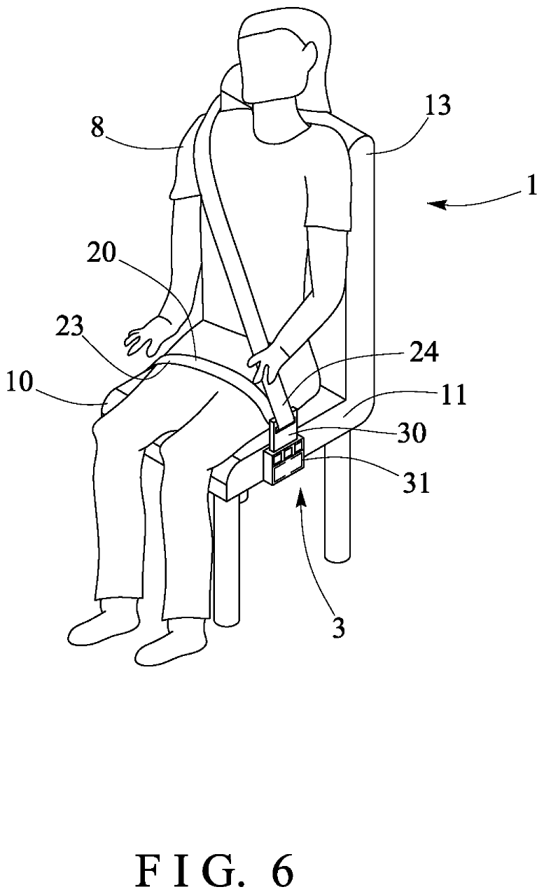

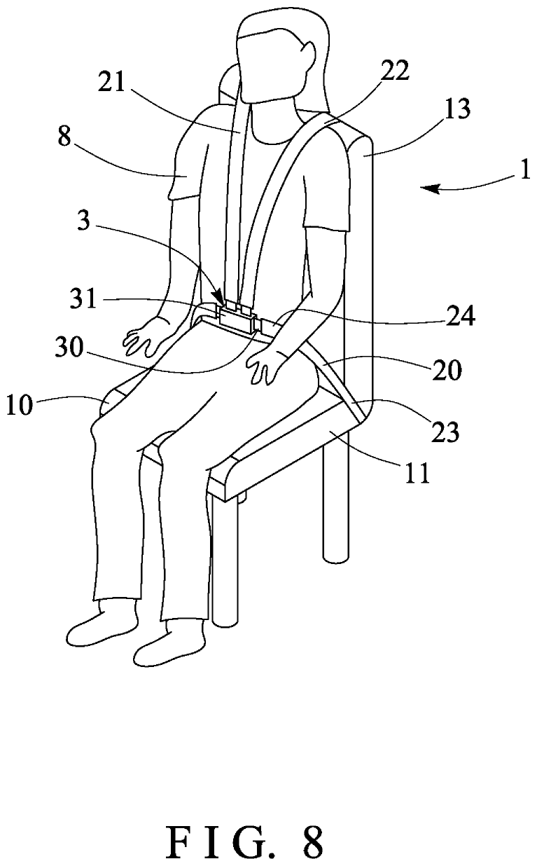

The lock device 3 includes a lock body or lock member 31 attached or mounted or secured to such as the first side portion 11 of the chair 10 or of the carrier device 1, and provided for engaging with the buckle member 30 and for solidly and stably anchoring or retaining or positioning or supporting the user 8 on the carrier device 1. As shown in FIGS. 1-4, the strap member 20, 21, 22 may be provided for engaging with various portions of the user 8, such as the legs or feet of the user 8, and/or the front and/or the shoulder portion of the user 8 (FIGS. 6, 8).

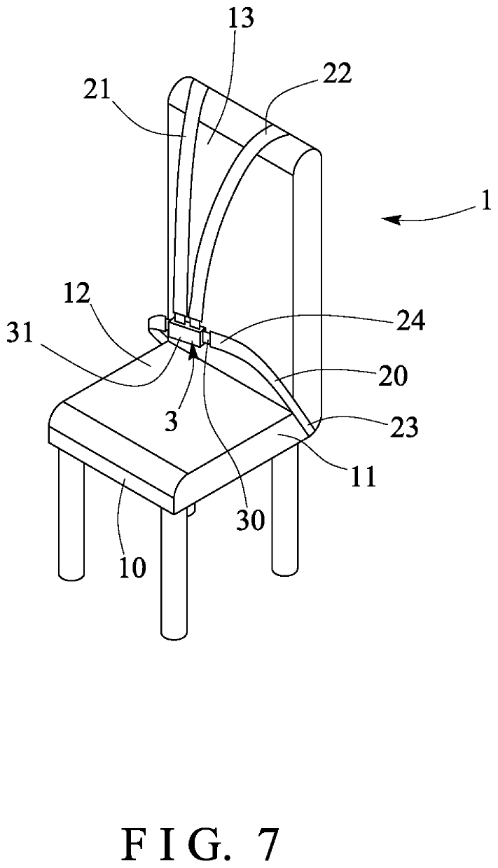

As shown in FIGS. 7 and 8, the lock member 31 of the lock device 3 may also be attached or mounted or coupled between the strap members 20, 21, 22. Alternatively, as shown in FIG. 9, the strap member 20, 21, 22 and the lock member 31 of the lock device 3 may also be attached or mounted or secured to the bed 15, for engaging with the legs or feet of the user 8 and for solidly and stably anchoring or retaining or positioning or supporting the user 8 on the bed 15 of the carrier device 1.

As shown in FIG. 10, a processing or controlling device 5 may further be provided for actuating or operating the belt device, for example, the controlling device 5 may be disposed or engaged into the lock member 31 of the lock device 3, and includes an input unit or module 50 for inputting or entering a value, such as a time interval, and electrically connected or coupled to a control unit or module 51, in which the control module 51 includes a timing unit or module 52 electrically connected or coupled to the input module 50 for receiving the time interval value from the input module 50.

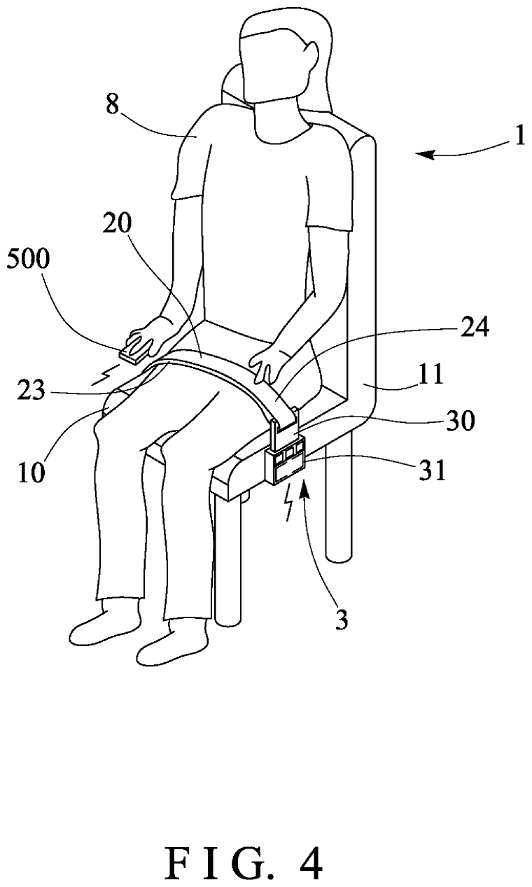

The control module 51 further includes a processing unit or module 53 electrically connected or coupled to the timing module 52 and/or electrically connected or coupled to the lock member 31 of the lock device 3 for locking or releasing the buckle member 30 from the lock member 31 selectively, and a remote unit or module 54 may further be provided and electrically connected or coupled to the processing module 53 for remote controlling the processing module 53 and/or the lock member 31. As shown in FIG. 4, an additional controlling device 500 may further be provided for remote controlling the processing module 53 and/or the lock member 31.

In operation, as shown in FIG. 10, the user 8 may input or enter a value, such as a time interval into the input module 50 of the controlling device 5, and the processing module 53 may receive the time interval value from the timing module 52 and may actuate or control the lock member 31 of the lock device 3, for example, the user 8 may input or enter a time interval value of about two (2) or more hours into the input module 50, and when the time interval value is reached, the processing module 53 may actuate or control the lock member 31 to release the buckle member 30 from the lock member 31 selectively and to warn that the user 8 has not been moved or has not exercised for a long time, and for reminding the user 8 to do some physical exercise.

Accordingly, the belt device combination in accordance with the present invention includes an improved and smart or intelligent structure or configuration for posture correction purposes and for reminding the user that he has kept a sedentary posture for a long time and that he is required to do some physical exercise.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A belt device combination comprising:
a carrier member for supporting a user,
a strap member for engaging with the user, said strap member including a first end portion attached to said carrier member, and a second end portion,
a lock member attached to said carrier member,
a buckle member attached to said second end portion of said strap member for engaging with said lock member and for anchoring the user to said carrier member, and
a controlling device attached to said lock member, said controlling device including a processing module connected to said lock member for releasing said buckle member from said lock member selectively, and a timing module connected to said processing module for controlling a releasing time interval of said buckle member from said lock member.

2. The belt device combination as claimed in claim 1, wherein said controlling device includes an input module connected to said timing module.

3. The belt device combination as claimed in claim 1, wherein said controlling device includes a remote module connected to said processing module.

4. The belt device combination as claimed in claim 1, wherein said carrier member is selected from a chair, said first end portion of said strap member is attached to a first side portion of said carrier member, and said lock member is attached to a second side portion of said carrier member.
